(19) United States
(12) Patent Application Publication
(10) Pub. No.: US 2019/0325998 A1
(43) Pub. Date: Oct. 24, 2019

(54) Enformedconsents

(71) Applicants: Kamran Hasni, London, KY (US); Mary Perkins, London, KY (US); Maria Swafford, Bryant's Station, KY (US)

(72) Inventors: Kamran Hasni, London, KY (US); Mary Perkins, London, KY (US); Maria Swafford, Bryant's Station, KY (US)

(73) Assignee: Kamran Hasni, London, KY (US)

(21) Appl. No.: 15/530,107

(22) Filed: Dec. 5, 2016

Related U.S. Application Data
(60) Provisional application No. 62/339,367, filed on May 20, 2016.

(51) Int. Cl. G16H 15/00; G16H 10/60

(52) U.S. Cl. CPC G16H 15/00; G16H 10/60

(57) ABSTRACT

Enformedconsent is a virtual, interactive electronic consent in which a patient receives information about a procedure such as risks, benefits, and options. The patient will also be able to view a video explaining the procedure in detail to the patient, as well as a FAQ section. The patient will be able to interact with the software by being able to ask questions, and receive answers. Once the patient has viewed the information, he or she may sign the electronic consent, the document is then uploaded to the electronic health record.

### CROSS REFERENCE TO RELATED APPLICATION

This application claims the benefit of U.S. Provisional Application No. 62339367 filed on 2016-12-05, entitled “Enformedconsent”

### BACKGROUND OF INVENTION

### Field of Invention

Enformedconsent is a virtual, interactive electronic consent in which a patient receives information about a procedure such as risks, benefits, and options. The patient will also be able to view a video explaining the procedure in detail to the patient, as well as a FAQ section. The patient will be able to interact with the software by being able to ask questions, and receive answers. Once the patient has viewed the information, he or she may sign the electronic consent.

### BRIEF DESCRIPTION OF DRAWINGS

FIG. 1; is a splash screen,

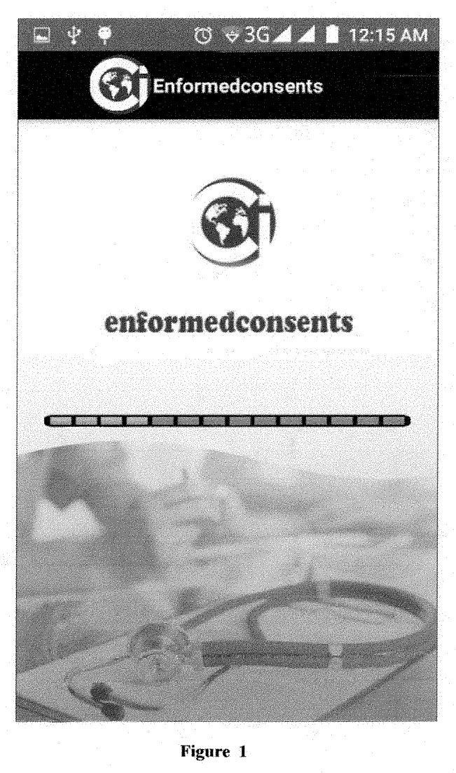

FIG. 2: Open the app by clicking on the launcher icon in android mobile. Fill all the field and press send. After pressing send button new activity will open.

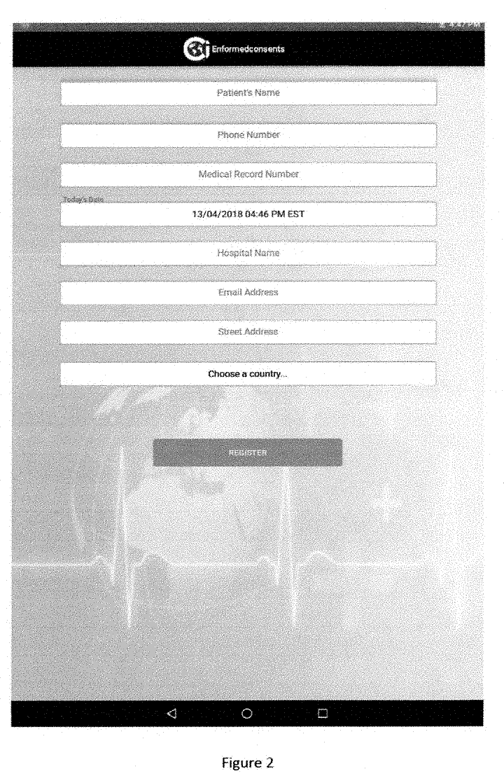

FIG. 3: Fill all the Field and press Send Button. After pressing Send Button New Activity will be open.

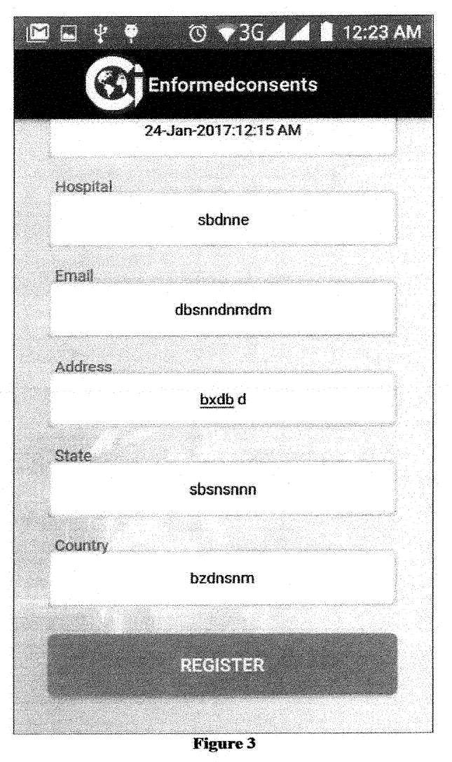

FIG. 4a. Now here is the next activity all the procedure will be list down Click the arrow icon if you want to show all the procedure

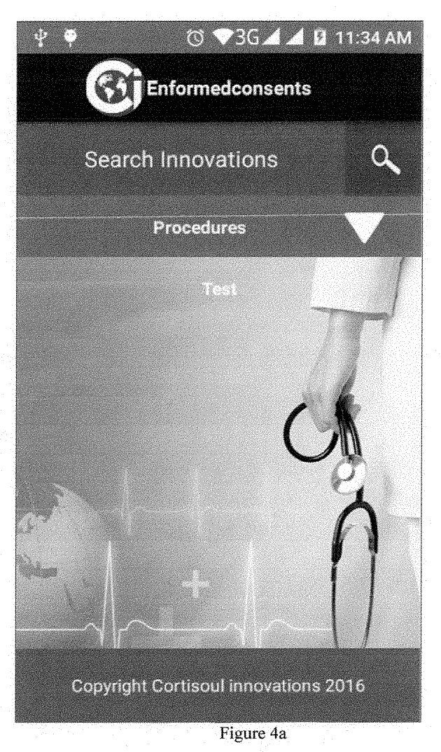

And also patient search procedure by its name, now select procedure, after

Selecting procedure next activity will be open.

FIG. 4b: In this activity the details of the procedure will be shown ,and also the sketch of cartoon read the full details of the procedure. Wait for the end of the details. when the cartoon sketch reach to the end of details new activity will be open.

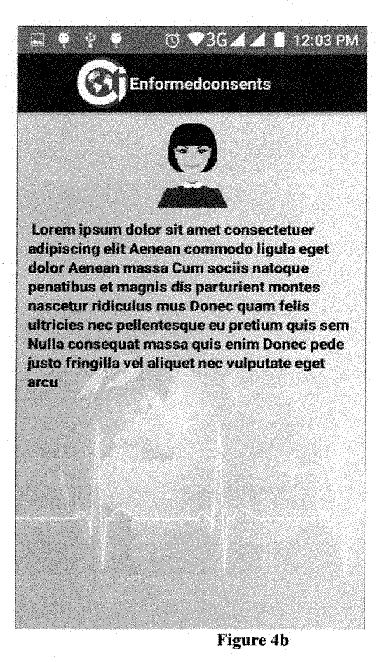

FIG. 5: In this activity the video of the procedure will be play. Press FAQ's Button if you want to see FAQ's related to this procedure

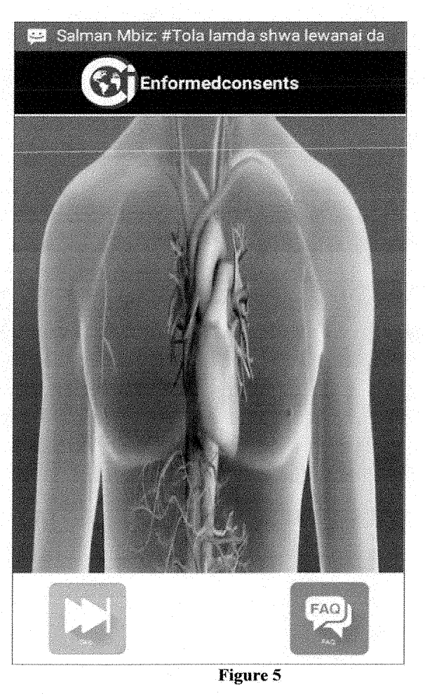

FIG. 6a: instructions on how to use program

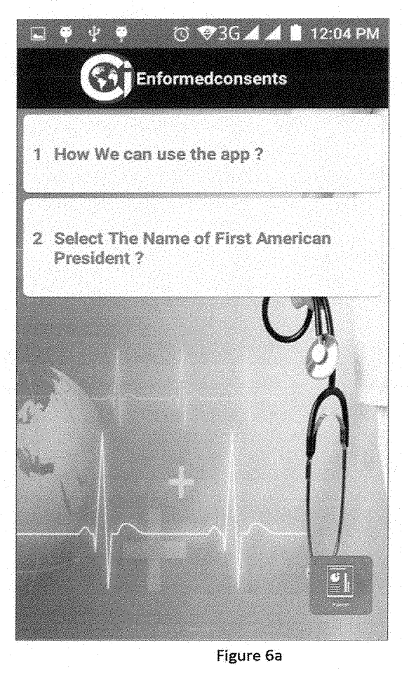

FIG. 6b: Press Skip Button if you want to submit the agreement, after clicking skip button Digital signature activity will be open.

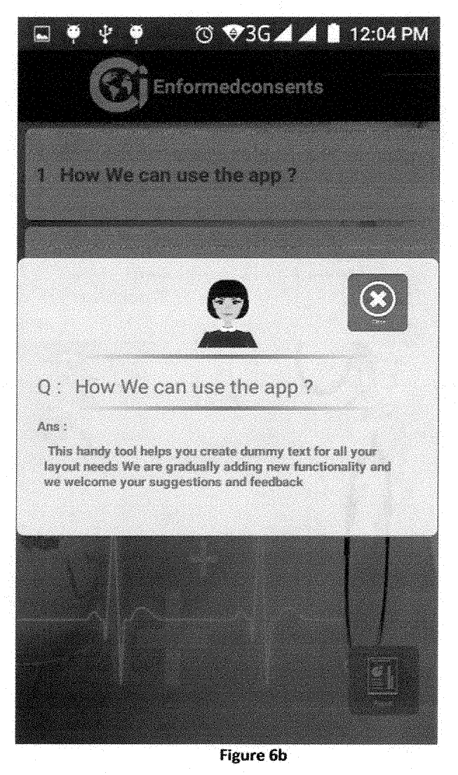

FIG. 7a: When you click on the sign button it will open the second activity, make signature and press save , also if you want to clear the signature in case of any wrong signature press the clear button.

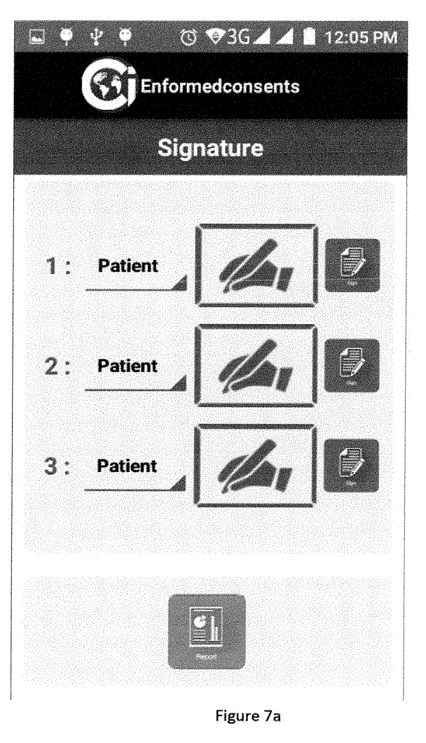

FIG. 7b: Patient signature page

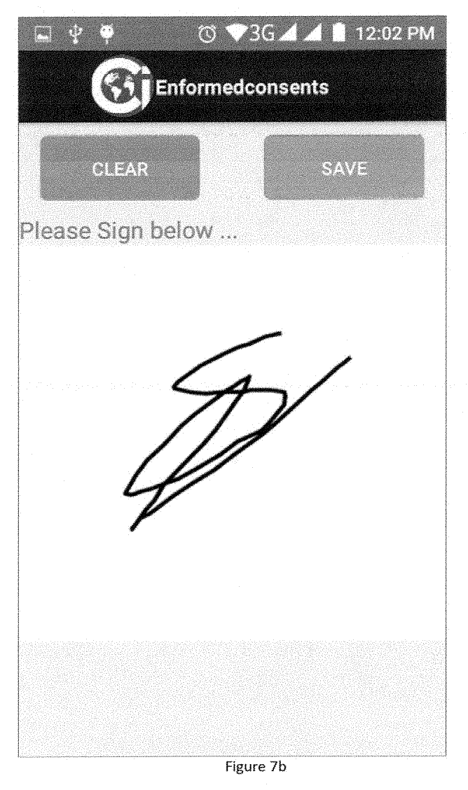

FIG. 8a: Signature page

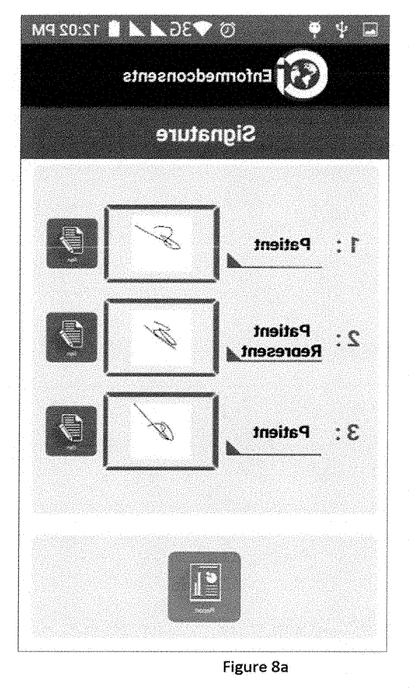

FIG. 8b: After Signature Click on Report Button to make Pdf Report of the info.

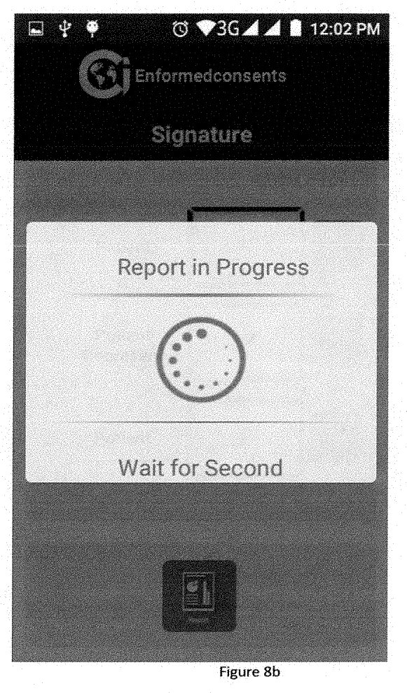

FIG. 9: File and upload page

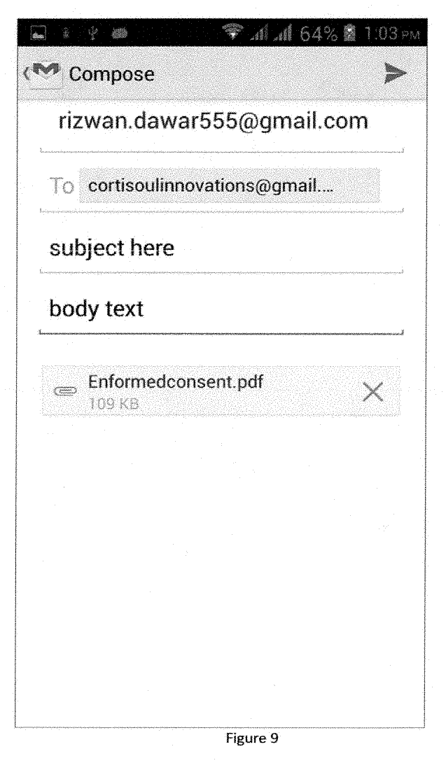

### BRIEF SUMMARY OF THE INVENTION

Enformedconsent is an electronic, virtual, and interactive software program that allows the patient to view a video about their procedure, show risks and benefits, as well as lets the patient ask questions that is sent to the physician. The physician can answer those questions when he or she meets with the patient. After the patient has addressed all concerns then he or she can sign the consent electronically. The signed consent and education document then can be uploaded to the consumer's electronic health record.

1. Enformedconsent is an electronic, virtual, and interactive software program that allows the patient to view a video about their procedure, show risks and benefits, as well as lets the consumer ask questions that can be sent to the physician.

2. After the patient has addressed all concerns then he or she can sign the consent electronically.

3. The signed consent and education document then can be uploaded to the consumer's electronic health record.
